(19) United States
(12) Patent Application Publication
(10) Pub. No.: US 2009/0143611 A1
(43) Pub. Date: Jun. 4, 2009

(54) Hexafluoroalcohol-based Monomers and Processes of Preparation Thereof

(75) Inventors: Shahrokh Motallebi, St-Lambert (CA); Denis Desilets, St-Jean-sur-Richelieu (CA); Ian Marcotte, Longueuil (CA); Luc Bouchard, La Prairie (CA); Khalil Feghali, St-Jean-sur-Richelieu (CA); Pushkara Rao Varanasi, Poughkeepsie, NY (US); Mahmoud Khojasteh, Poughkeepsie, NY (US)

Correspondence Address: BACHMAN & LAPOINTE, P.C., 900 CHAPEL STREET, SUITE 1201, NEW HAVEN, CT, 06510, US

(73) Assignees: ST-JEAN PHOTOCHIMIE INC., Quebec (CA); INTERNATIONAL BUSINESS MACHINES CORPORATION, Armonk, NY (US)

(21) Appl. No.: 11/719,138

(22) PCT Filed: Nov. 14, 2005
(86) PCT No.: PCT/CA05/01728

§ 371 Date: Oct. 28, 2007

Related U.S. Application Data
(60) Provisional application No. 60/627,019, filed on Nov. 12, 2004.

(51) Int. Cl. C07C 69/74

(52) U.S. Cl. 560/118; 560/116

(57) ABSTRACT

The present invention relates to compounds of formula (I): wherein R₁ is H, or a C₁-C₁₀ linear, branched or cyclic alkyl group which is unsubstituted or substituted with fluorine; R₂ is an alicyclic group having 5 to 20 carbon atoms which is unsubstituted or substituted with fluorine; and R₃ represents a C₁-C₁₀ linear or branched alkylene which is unsubstituted or substituted with fluorine. Processes for preparing such compounds are also disclosed. The compounds of the present invention can be used as monomers in the fields of photolithography and semiconductor fabrication.

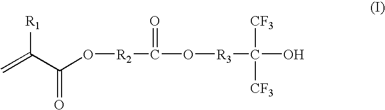

### TECHNICAL FIELD

The present invention is related to the fields of photolithography and semiconductor fabrication. More specifically, this invention relates to novel compounds or monomers comprising a hexafluoroalcohol moiety. These compounds are particularly useful in the preparation of base resins used for chemical amplification resist compositions suited for microfabrication.

### BACKGROUND OF THE INVENTION

Trends in the photolithography and semiconductor industry continually require higher circuit density in microelectronic substrates which are made from ultrafine pattern consisting of lines having a width of 0.25 microns or less.

For imaging very fine features at the submicron level in semiconductor devices, higher photoimaging resolution is necessary. In order to achieve this, photoresist material is irradiated using electromagnetic radiation in the far or extreme ultraviolet region (193 nm or less).

However, wavelength of 193 nm or less correspond to the absorption maximum of the aromatic rings and therefore photoresist materials containing aromatic rings so far used by industry become inadequate and useless for application within these wavelength ranges.

Thus, with changes in the exposure wavelength, photoresist compositions have to be modified and therefore new classes of compounds having different structures and photosensitivity mechanisms should be introduced.

In recent years, various resist materials having transparency to short wavelength imaging radiation with sufficient resistance to a reactive etching processing environment have been developed. Among these materials, acrylic polymers containing alicyclic structures have been proposed.

These acrylic polymers attracted attention since they were easily prepared and also in view of their transparency at short wavelength (193 nm or less). They also have been found to have an interesting etch resistance essentially due to alicyclic structures such as adamantanes, norbornanes, and the like.

Unfortunately, these polymers exhibit poor solubility in an alkaline aqueous solution and also poor adhesion to the substrate due to hydrophobic properties of the polymer. In order to overcome these problems, there have been some reports of polar hydroxyl- and fluorine-containing norbornene materials.

However, with respect to a resist material of a sufficient transparency at wavelength of 193 nm or less and etch resistance, there is a growing need for new materials having improved performances such as superior pattern contrast (change in solubility upon irradiation) and improved adhesive strength to the substrate.

Thus, there is clearly a need for novel compounds which can permit to overcome the above-mentioned drawbacks.

### SUMMARY OF THE INVENTION

It is therefore an object of the present invention to provide compounds that can be useful as polymerizable monomers which can be used in the preparation of polymers having a superior pattern contrast.

It is also another object of the present invention to provide compounds that can be used as polymerizable monomers in the preparation of high transparency polymers.

It is also another object of the present invention to provide compounds that can be used as polymerizable monomers in the preparation of polymers having a high etching resistance and an excellent resolution and sensitivity.

It is also another object of the present invention to provide compounds that can be used as polymerizable monomers in the preparation of polymers which have an improved adhesion to a substrate.

It is also another object of the present invention to provide compounds which could be produced at low costs.

It is also another object of the present invention to provide a process for preparing the previously mentioned compounds.

According to one aspect of the invention there is provided a compound of formula (I):

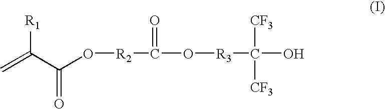

wherein

R₁ is H, or a C₁-C₁₀ linear, branched or cyclic alkyl group which is unsubstituted or substituted with fluorine;

R₂ is an alicyclic group having 5 to 20 carbon atoms which is unsubstituted or substituted with fluorine; and

R₃ represents a C₁-C₁₀ linear or branched alkylene which is unsubstituted or substituted with fluorine.

It has been found that the compounds of the present invention upon polymerization, can provide polymers of high transparency, etching resistance and excellent resolution and sensitivity.

According to another aspect of the invention there is provided a process for preparing a compound of formula (I):

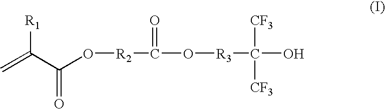

wherein

R₁ is H, or a C₁-C₁₀ linear, branched or cyclic alkyl group which is unsubstituted or substituted with fluorine;

R₂ is an alicyclic group having 5 to 20 carbon atoms which is unsubstituted, or substituted with fluorine; and

R₃ represents a C₁-C₁₀ linear or branched alkylene which is unsubstituted or substituted with fluorine,

comprising the step of reacting together a compound of formula (IV) and a compound of formula (V):

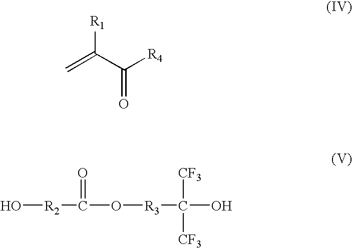

wherein

R₁, R₂, and R₃ are as previously defined; and

R₄ is
Cl, Br, I, imidazolyl, OR₅ or

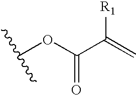

and wherein R₅ is H, or a C₁-C₆ branched or linear alkyl.

According to another aspect of the invention there is provided a process for preparing a compound of formula (IX):

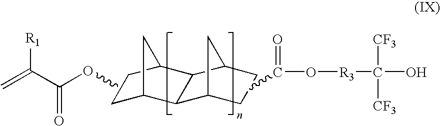

wherein

R₁ is H, or a C₁-C₁₀ a linear, branched or cyclic alkyl group which is unsubstituted or substituted with fluorine.

R₃ represents a C₁-C₁₀ linear or branched alkylene which is unsubstituted or substituted with fluorine.

n has a value of 0 or 1,

comprising the steps of

a) reacting together a compound of formula (VII) and a compound of formula (VIII) so as to obtain a compound of formula (VI):

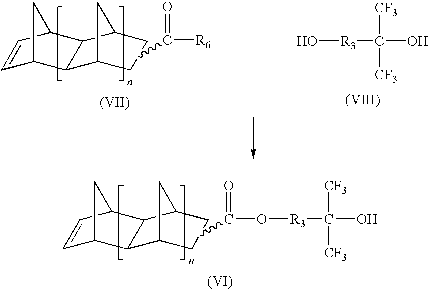

wherein

n and R₃ are as previously defined;

R₆ is Cl, Br, I, imidazolyl or OR₇, R₇ being H, a C₁-C₆ branched or linear alkyl

b) hydroxylating the compound of formula (VI) so as to obtain a compound of formula (X);

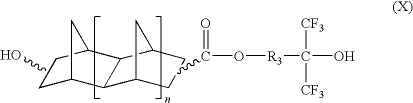

wherein n and R₃ are as previously defined

c) reacting the compound of formula (X) together with a compound of formula (IV)

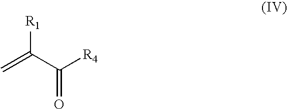

wherein

R₁ is as previously defined;

R₄ is

Cl, Br, I, imidazolyl, OR₅ or

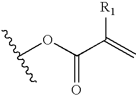

wherein

R₁ is as previously defined;

R₅ is a C₁-C₆ branched or linear alkyl,

so as to obtain said compound of formula (IX).

It has been found that the processes of the present invention permit to efficiently and economically prepare the previous mentioned compounds. It has also been found that these processes can be applied to large or industrial scale.

The expression “alicyclic group having 5 to 20 carbon atoms” as used herein refers to a C₅-C₂₀ cycloaliphatic group which is monocyclic or polycyclic. The cycloaliphatic monocyclic group is preferably cyclopentyl or cyclohexyl. The cycloaliphatic polycyclic group is preferably norbornyl, adamantyl or tricyclo[5.2.1.0]decanyl.

In the compounds and processes of the present invention R₁ is preferably H or CH₃. R₂ is preferably a group of formula (II):

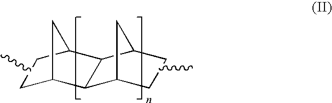

wherein n has a value of 0 or 1, and wherein said R₂ group is unsubstituted or substituted with fluorine. The person skilled in the art will understand from formula (II) that the latter can be substituted by one or more fluorine atoms at the positions where there is a hydrogen atom.

Alternatively, R₂ can be a group of formula (III):

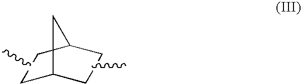

R₂ can also be of formula (XI):

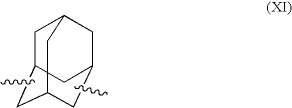

The compounds of formulas (III) and (XI) can also be substituted by a fluorine atom as previously indicated for formula (II).

In the compounds and processes of the present invention R₃ is preferably an unsubstituted C₃-C₇ linear or branched alkylene.

In the process for preparing a compound of formula (I), the compounds of formulas (IV) and (V) are preferably reacted together in the presence of a base and/or a coupling reagent. Similarly, in the process for preparing a compound of formula (IX), the compounds of formulas (VII) and (VIII) are preferably reacted together in the presence of a base and/or a coupling reagent. The base can be triethylamine, EtN-iPr₂, 4-dimethylaminopyridine, NaHCO₃, Na₂CO₃, KHCO₃, K₂CO₃, Cs₂CO₃, 2,6-lutidine, 1,8,diazabicyclo[5.4.0]undec-7-ene (DBU), Li₂CO₃ etc. The coupling reagent can be 1,3-dicyclohexylcarbodiimide (DCC), 1-cyclohexyl-3-(2-morpholinoethyl)carbodiimide metho-p-toluenesulfonate, 1-ethyl-3-(3′-dimethylaminopropyl)carbodimide hydrochloride (EDCl), (CH₃)₃SiCl, (CH₃)₂SiCl₂, CH₃SiCl₃, SiCl₄, isopropenyl chloroformate, TiCl₄, AgClO₄, benzotriazol-1-yloxytris(dimethylamino)phosphonium Hexafluorophosphate (BOP), O-benzotriazol-1-yl-N,N,N′,N′-tetramethyluronium hexafluoro-phosphate (HBTU), bis(2-oxo-3-oxazolidinyl)phosphinic chloride (BOP-Cl) etc.

In the process for preparing a compound of formula (I), R₂ is preferably a group of formula (II):

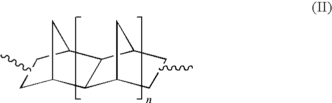

where n has a value of 0 or 1, and wherein said compound of formula (V) can be prepared by hydroxylating a compound of formula (VI):

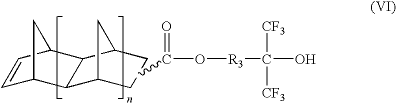

n and R₃ being as previously defined.

Preferably, the hydroxylation is carried out using a borane-based reagent and an oxidizer. The borane-based reagent can be BH₃, disiamylborane, 9-borabicyclo[3.3.1]nonane, catecholborane, thexylborane, thexylchloroborane, dibromoborane, diisopinocamphenylborane etc. The oxidizer can be NaOH/H₂O₂, trimethylamine N-oxide etc. These preferred embodiments concerning the hydroxylation are also suitable for the process for preparing the compound of formula (IX).

The compound of formula (VI) can be prepared by reacting together a compound of formula (VII) and a compound of formula (VIII)

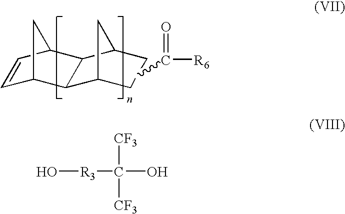

wherein

n and R₃ are as previously defined; and

R₆ is Cl, Br, I, imidazolyl or OR₇, R₇ being H, a C₁-C₆ branched or linear alkyl.

The compounds of formulas (VII) and (VIII) are preferably reacted together in the presence of a base and/or a coupling reagent. The base or the coupling reagent are preferably the same than those previously mentioned.

In the process for preparing a compound of formula (IX), step (a) and/or step (c) can be carried out in the presence of at least one of a base and a coupling agent. The base or the coupling reagent are preferably the same than those previously mentioned.

The following examples are three compounds which are particularly preferred in the compounds and processes of the invention.

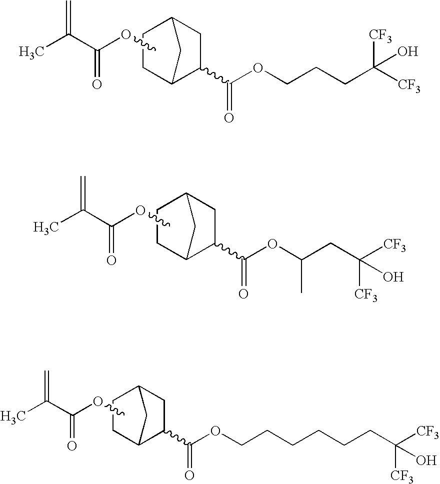

### DESCRIPTION OF PREFERRED EMBODIMENTS

Further features and advantages of the invention will become more readily apparent from the following description of preferred embodiments as illustrated by way of examples.

The following non-limiting examples further illustrate the invention. In particular, some preferred embodiments concerning the compounds and processes of the invention are more specifically described.

### EXAMPLE 1

### Preparation of 2-(6-Chloro-hexyloxy)-tetrahydro-pyran (1)

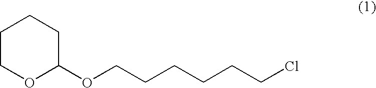

A mixture of 6-chlorohexan-1-ol (180 g, 1.32 mol), p-toluenesulfonic acid monohydrate (12.5 g, 0.08 mol) and dichloromethane (600 g) was stirred for 30 minutes. The 3,4-dihydro-2H-pyran (133 g, 1.58 mol) was added in 35 minutes. The solution was stirred for 15 hours at room temperature. The dark yellow solution was washed with 10% aqueous NaOH (2×200 mL) and with water (2×150 mL). The solvent was evaporated. The dark oil was purified by distillation under reduced pressure to give 217 g of colorless oil (yield=74%).

### EXAMPLE 2

### Preparation of 1,1,1-Trifluoro-8-(tetrahydro-pyran-2-yloxy)-2-trifluoromethyl-octan-2-ol (2)

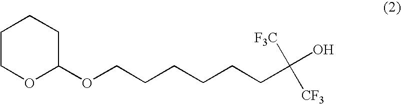

A flame dried 250 mL flask was charged with magnesium turnings (9.51 g, 0.39 mol) and anhydrous THF (100 mL). Red-Al® 65+ wt. % solution was added (2 mL). 2-(6-Chloro-hexyloxy)-tetrahydro-pyran 1 was added in 1 hour. The mixture was refluxed during 3 hours and cooled at room temperature. Hexafluoroacetone (47.25 g, 0.28 mol) was condensed with Dewar type condenser (Acetone/Dry ice) in a 500 mL round bottom flask cooled at −78° C. Anhydrous THF (200 mL) was added to hexafluoroacetone containing flask. The Grignard's reagent solution was added slowly to the cold hexafluoacetone solution. The mixture was sticky and the cooling bath was removed. The solution was stirred overnight at room temperature. The mixture was acidified to obtain pH=6. The aqueous layer was extracted with MTBE (methyl tert-butyl ether) (3×50 mL). The MTBE layer was washed with 5% NaHCO₃ (2×50 mL) and with water (100 mL), dried (MgSO₄) and concentrated. The residue was purified by chromatography on silica gel (Ethyl Acetate/Hexane 1/9 v/v) to give 57 g (yield=58%) of clear oil.

### EXAMPLE 3

### Preparation of 8,8,8-Trifluoro-7-trifluoromethyl-octane-1,7-diol (3)

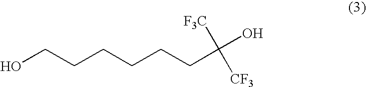

A 500 mL flask was charged with 1,1,1-Trifluoro-8-(tetrahydro-pyran-2-yloxy)-2-trifluoromethyl-octan-2-ol (2) (17.4 g, 49.39 mmol), p-toluenesulfonic acid monohydrate (0.76 g, 4.00 mmol) and methanol (380 mL). The solution was heated at 45° C. for 3 hours and cooled at room temperature. Water (1000 mL) was added. The solution was extracted with MTBE (3×250 mL). The ether layer was washed with brine, dried (MgSO₄) and concentrated to give 13 g (yield=98%) of yellowish oil. The product was used without purification.

### EXAMPLE 4

### Preparation of 5,5,5-Trifluoro-4-trifluoromethyl-pentane-1,4-diol (4) and 1,1,1-Trifluoro-2-trifluoromethyl-pentane-2,4-diol (5)

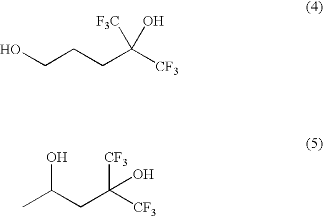

To a stirred and cooled solution of 1,1,1-Trifluoro-2-trifluoromethyl-pent-4-en-2-ol (15 g, 72 mmol) in dry tetrahydrofuran (150 mL), Borane-tetrahydrofuran complex (1.0M solution in tetrahydrofuran, 120 mL, 120 mmol) was added dropwise under nitrogen atmosphere. The mixture was then stirred at room temperature for 30 minutes and then cooled to 0-5° C. A solution of sodium hydroxide (3.0M, 70 mL) was added dropwise over 30 minutes period followed by addition of hydrogen peroxide (30%, 27 mL) over a period of 20-30 minutes. The resulting mixture was stirred at room temperature for another 1.0 hour and water (100 mL) was added. The mixture was acidified to PH 4-5 and the product was extracted with methylene chloride (3×200 mL). The methylene chloride extract was dried with magnesium sulfate, filtered and evaporated under reduced pressure to yield 14 g of the mixture of products (4) and (5). The resulting crude product was purified by chromatography on silica gel (tetrahydrofuran/Hexane 3/6 v/v) to give 6 g of (4) and 7 g of (5) as colorless oils.

### EXAMPLE 5

### Preparation of Bicyclo[2.2.1]hept-5-ene-2-carboxylic acid 8,8,8-trifluoro-7-hydroxy-7-trifluoromethyl-octyl ester (6)

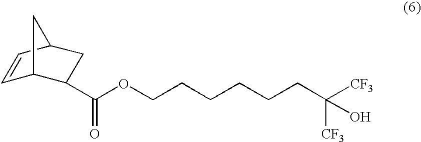

To a solution of 5-norbornene-2-carboxylic acid (3.72 g, 26.95 mmol) and dichloromethane (30 g), oxalyl chloride (4.45 g, 35.04 mmol) was added dropwise under nitrogen atmosphere at room temperature. The mixture was stirred at this temperature for two hours and the volatiles were then removed under reduced pressure. To the resulting oil, a solution of 8,8,8-Trifluoro-7-trifluoromethyl-octane-1,7-diol 3 (6.0 g, 22.37 mmol) and dichloromethane (60 g) was added and the mixture was cooled to 0° C. A solution of triethylamine (2.72 g, 26.95 mmol), 4-(dimethylamino)pyridine (0.53 g, 4.34 mmol) in dichloromethane (10 g) was then added over a 15 minutes period. The resulting mixture was stirred at room temperature for 1.0 hour, acidified to pH 4-5, washed with water (3×100 mL), dried over magnesium sulfate and filtered. After removal of dichloromethane under reduced pressure, 7.9 g of colourless oil was obtained. The resulting oil was purified by chromatography on silica gel (Ethyl acetate/Hexane 2/8 v/v) to give 7 g of colorless oil.

### EXAMPLE 6

### Preparation of Bicyclo[2.2.1]hept-5-ene-2-carboxylic acid 5,5,5-trifluoro-4-hydroxy-4-trifluoromethyl-pentyl ester (7)

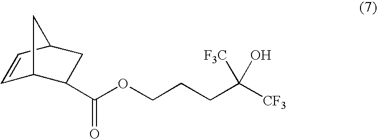

This product was prepared from the alcohol (4) of example 4 using the same procedure described in example 5. The product was used without purification.

### EXAMPLE 7

### Preparation of Bicyclo[2.2.1]hept-5-ene-2-carboxylic acid 4,4,4-trifluoro-3-hydroxy-1-methyl-3-trifluoromethyl-butyl ester (8)

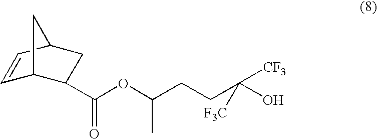

This product was prepared from the alcohol (5) of example 4 using the same procedure described in example 5. The product was purified on silica gel (ethyl acetate/Hexane 1/9 v/v) to give a colorless oil.

### EXAMPLE 8

### Preparation of Compound (9)

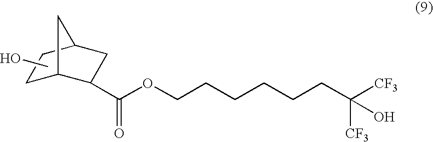

To a cooled (0° C.) and stirred solution of Bicyclo[2.2.1]hept-5-ene-2-carboxyl ic acid 8,8,8-trifluoro-7-hydroxy-7-trifluoromethyl-octyl ester (6) (22.3 g, 53.0 mmol) and tetrahydrofuran (250 mL), borane-tetrahydrofuran complex (1.0M solution in tetrahydrofuran, 160 mL, 160 mmol) was added dropwise under nitrogen atmosphere. The resulting mixture was then stirred at room temperature for 1.0 hour. The solution was cooled again to 0° C. and a solution of sodium hydroxide (3.0M, 52 mL) was added dropwise over 30 minutes period followed by addition of hydrogen peroxide (30%, 48 mL) over a period of 20-30 minutes. The resulting mixture was stirred at room temperature for another 2.0 hours and water (100 mL) was added. The mixture was acidified to PH 4-5 and the product was extracted with methylene chloride (3×200 mL). The methylene chloride extract was dried with magnesium sulfate, filtered and evaporated to yield 21.7 g of oil. The resulting crude product was purified by chromatography on silica gel (Ethyl acetate/Hexane 1/9 v/v) to give 12.5 g of (9) as colorless oil.

### EXAMPLE 9

### Preparation of Compound (10)

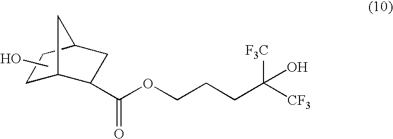

This product was prepared from the alcohol (7) of example 6 using the same procedure described in example 8. The thus obtained colorless oil was used without purification.

### EXAMPLE 10

### Preparation of Compound (11)

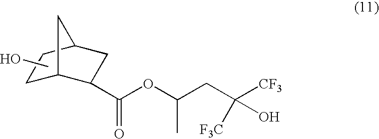

This product was prepared from the alcohol (8) of example 7 using the same procedure described in example 8. The thus obtained colorless oil was used without purification.

### EXAMPLE 11

### Preparation of Compound (12)

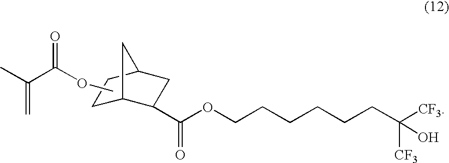

To a stirred solution of (9) (9.26, 23.0 mmol), methacrylic anhydride (4.2 g, 27 mmol) and tetrahydrofuran (44 g), was added a solution of 4-(dimethylamino) pyridine (3.3 g, 27 mmol) in tetrahydrofuran (20 g) over a period of 30 minutes under nitrogen atmosphere. The resulting mixture was stirred for a further 2.0 hours at room temperature. The mixture was then acidified, extracted with dichloromethane (500 mL), washed, dried over magnesium sulfate and filtered. After removal of dichloromethane under reduced pressure, the resulting crude product was purified by chromatography on silica gel (Ethyl acetate/Hexane) to give 5.5 g of (12) as colorless oil.

### EXAMPLE 12

### Preparation of Compound (13)

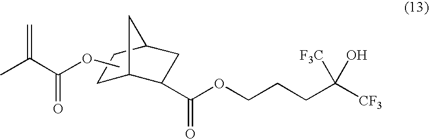

This product was prepared from the alcohol (10) of example 9 using the same procedure described in example 11. The product was purified by chromatography on silica gel (MTBE/Hexane) to provide a colorless oil.

### EXAMPLE 13

### Preparation of Compound (14)

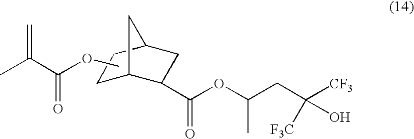

This product was prepared from the alcohol (11) of example 10 using the same procedure described in example 11. The product was purified by chromatography on silica gel (MTBE/Hexane) to provide a colorless oil.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A compound of formula (I): 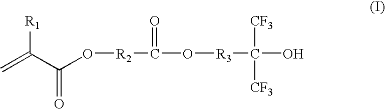
wherein
R₁ is H, or a C₁-C₁₀ linear, branched or cyclic alkyl group which is unsubstituted or substituted with fluorine;
R₂ is an alicyclic group having 5 to 20 carbon atoms which is unsubstituted or substituted with fluorine; and
R₃ represents a C₁-C₁₀ linear or branched alkylene which is unsubstituted or substituted with fluorine.

2. The compound of claim 1, wherein R₁ is H or CH₃.

3. The compound of claim 1, wherein R₂ is a group of formula (II): 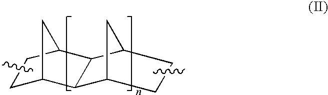
wherein n has a value of 0 or 1, and wherein said R₂ group is unsubstituted or substituted with fluorine.

4. The compound of claim 1, wherein R₂ is unsubstituted and represents a group of formula (III):

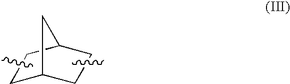

5. The compound of claim 1, wherein R₃ is an unsubstituted C₃-C₇ linear or branched alkylene.

6. The compound of claim 1 having the following formula:

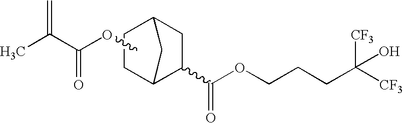

7. The compound of claim 1 having the following formula:

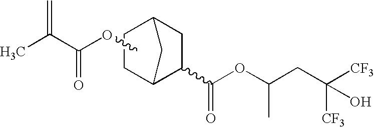

8. The compound of claim 1 having the following formula:

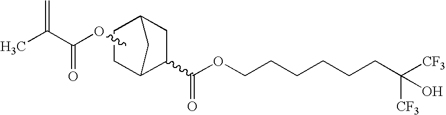

9. A process for preparing a compound of formula (I): 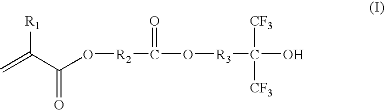
wherein
R₁ is H, or a C₁-C₁₀ linear, branched or cyclic alkyl group which is unsubstituted or substituted with fluorine;
R₂ is an alicyclic group having 5 to 20 carbon atoms which is unsubstituted, or substituted with fluorine; and
R₃ represents a C₁-C₁₀ linear or branched alkylene which is unsubstituted or substituted with fluorine,
comprising the step of reacting together a compound of formula (IV) and a compound of formula (V): 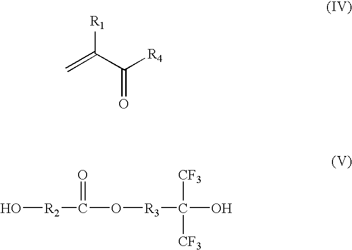
wherein
R₁, R₂, and R₃ are as previously defined; and
R₄ is
Cl, Br, I, imidazolyl, OR₅ or 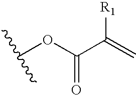
and wherein R₅ is H, a C₁-C₆ branched or linear alkyl.

10. The process of claim 9, wherein the compounds of formulas (IV) and (V) are reacted together in the presence of a base and/or a coupling reagent.

11. The process of claim 9, wherein R₂ is a group of formula (II): 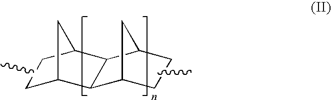
where n has a value of 0 or 1, and wherein said compound of formula (V) is prepared by hydroxylating a compound of formula (VI): 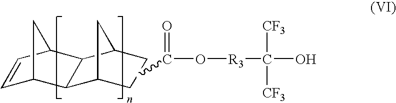
n and R₃ being as previously defined.

12. The process of claim 11, wherein said hydroxylation is carried out using a borane-based reagent and an oxidizer.

13. The process of claim 12, wherein said borane-based reagent is BH₃, disiamylborane, 9-borabicyclo[3.3.1]nonane, 9-borabicyclo[3.3.1]nonane, catecholborane, thexylborane, thexylchloroborane, dibromoborane or diisopinocamphenylborane.

14. The process of claim 12, wherein said oxidizer is NaOH/H₂O₂ or trimethylamine N-oxide.

15. The process of claim 11, wherein said compound of formula (VI) is prepared by reacting together a compound of formula (VII) and a compound of formula (VIII): 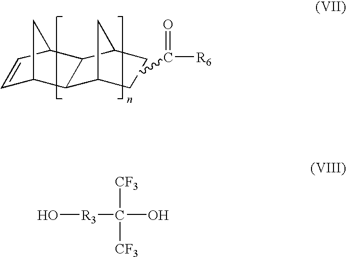
wherein
n and R₃ are as previously defined; and
R₆ is Cl, Br, I, imidazolyl or OR₇, R₇ being H, a C₁-C₆ branched or linear alkyl.

16. The process of claim 15, wherein compounds of formulas (VII) and (VIII) are reacted together in the presence of a base and/or a coupling reagent.

17. The process of claim 9, wherein R₁ is H or CH₃.

18. The process of claim 9, wherein R₂ is a group of formula (II): 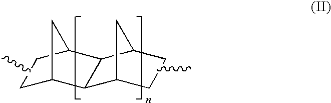
wherein n has a value of 0 or 1, and wherein said R₂ group is unsubstituted or substituted with fluorine.

19. The process of claim 9, wherein R₂ is unsubstituted and represents a group of formula (III):

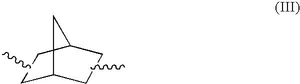

20. The process of claim 9, wherein R₃ is an unsubstituted C₃-C₇ linear or branched alkylene.

21. A process for preparing a compound of formula (IX): 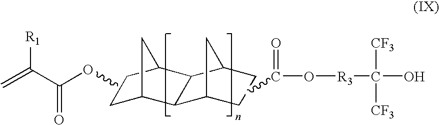
wherein
R₁ is H, or a C₁-C₁₀ a linear, branched or cyclic alkyl group which is unsubstituted or substituted with fluorine;
R₃ represents a C₁-C₁₀ linear or branched alkylene which is unsubstituted or substituted with fluorine; and
n has a value of 0 or 1,
comprising the steps of
a) reacting together a compound of formula (VII) and a compound of formula (VIII) so as to obtain a compound of formula (VI): 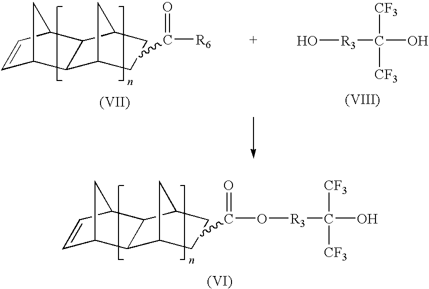
wherein
n and R₃ are as previously defined; and
R₆ is Cl, Br, I, imidazolyl or OR₇, R₇ being H, a C₁-C₆ branched or linear alkyl
b) hydroxylating the compound of formula (VI) so as to obtain a compound of formula (X); 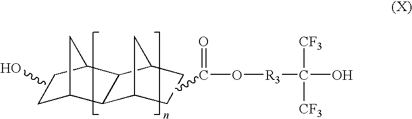
wherein n and R₃ are as previously defined
c) reacting the compound of formula (X) together with a compound of formula (IV) 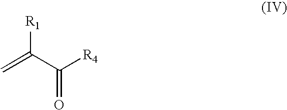
wherein
R₁ is as previously defined; and
R₄ is
Cl, Br, I, imidazolyl, OR₅ or 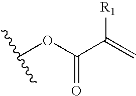
wherein
R₁ is as previously defined; and
R₅ is a C₁-C₆ branched or linear alkyl,
so as to obtain said compound of formula (IX).

22. The process of claim 21, wherein step (a) is carried out in the presence of at least one of a base and a coupling agent.

23. The process of claim 21, wherein step (c) is carried out in the presence of at least one of a base and a coupling agent.
